(19) United States
(12) Patent Application Publication
(10) Pub. No.: US 2008/0317859 A1
(43) Pub. Date: Dec. 25, 2008

(54) Stabilization of Testosterone Within Transdermal Devices

(75) Inventors: Michel Sournac, Toulouse (FR); Joel Bougaret, Francarville (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: PIERRE FABRE MEDICAMENT, BOULOGNE (FR)

(21) Appl. No.: 12/086,964

(22) PCT Filed: Dec. 26, 2006
(86) PCT No.: PCT/FR2006/002873

§ 371 Date: Jun. 23, 2008

(30) Foreign Application Priority Data
Dec. 29, 2005 (FR) 0513435

(51) Int. Cl. A61K 9/00; A61K 31/568

(52) U.S. Cl. 424/484; 514/178

(57) ABSTRACT

The present invention relates to the chemical stabilization of testosterone contained in self-adhesive transdermal devices by way of the association of a desiccant agent with said device within a sealing packaging. The use of a desiccant agent makes it possible to limit the chemical degradation of the testosterone to androstenedione and other impurities and accordingly ensures storage of said device over periods of up to thirty-six months.

The present invention relates to the field of self-adhesive transdermal devices (SATDs) for the administration of testosterone and/or at least one derivative and especially to the stabilization of the testosterone contained in such devices, particularly when the testosterone is present in high concentrations.

Such SATDs are pharmaceutical forms enabling the percutaneous administration of active substances in the context of chronic pathologies, especially as a preventive measure. Accordingly, in the context of the administration of male hormones, SATDs are available which promote the administration of such hormones, especially testosterone.

Testosterone, known according to systemic nomenclature as 17-hydroxyandrost-7-en-3-one, is the main circulating hormone of the androgenic type. Under the action of 5-alpha-reductase, it is converted to dihydrotestosterone, the hormone responsible for sexual differentiation.

SATDs include reservoir devices and matrix devices. In the first case, the active substance is contained in a gel provided between a support and a membrane. In the second case, the active substance is contained within a matrix layer, which usually is itself self-adhesive.

One of the problems encountered with SATDs is the slow penetration of the active substance through the skin. In the case of testosterone replacement therapy it is possible to use absorption promoters and/or to create testosterone supersaturation conditions so as to bring about forced passive diffusion.

In the latter case, when the testosterone is in a state of supersaturation within the matrix, problems of stability of the testosterone in said matrix are encountered. Supersaturation with testosterone can in fact give rise to phenomena of testosterone crystallization, which consequently reduces percutaneous delivery. In addition, when SATDs containing testosterone in a state of supersaturation are stored for long periods of time ranging from several months to several years, the presence of hydroxylation products of that testosterone, such as androstenedione, and various oxidation impurities may be observed. After their production, the SATDs are in fact usually kept and stored whilst awaiting use by the patient. It is always desirable to have a product having a limit date for use which is as long as possible, in both an industrial and commercial context but also from the point of view of the patient or of the pharmacist providing said product. A permitted maximum androstenedione level of 3% by weight relative to the amount of testosterone and a level of other impurities of 3% by weight relative to the amount of testosterone, at the end of the shelf-life, are indispensable prerequisites for meeting the conditions for registration of a commercially viable product.

In certain cases, and more especially that described in WO9915156, the stability of testosterone in an SATD is obtained by the concomitant presence of a very small amount of another steroid hormone, estrone, in the self-adhesive matrix layer. The presence of estrone is not desirable in the context of a hormone therapy medical application. In other cases, and more especially WO0074933, the SATD is composed of a plurality of layers, one of which layers, being composed of polymers of the PVA, PVP or polyvinyl acetate type, is intended to absorb ambient moisture. The manufacture of such a device is relatively complex.

From this general observation it accordingly emerges that it is necessary to have a simple and low-cost means of protecting the testosterone present within SATDs, especially SATDs of the matrix type, in order to limit its degradation, by hydroxylation or by oxidation, this being the case over periods ranging from several months to several years. Known means for protecting the testosterone include the addition of antioxidants; however, although this limits the amount of androstenedione, other, unidentified impurities have been found to appear nonetheless. The addition of antioxidants within an adhesive matrix moreover presents problems relating to physiological compatibility, taking into account the risks of their passing through the skin. Consideration has been given to packaging under an inert atmosphere in association with a packaging material that is impermeable to oxygen. However, such a solution is only partially satisfactory in view of the costs involved and the fact that, over storage periods of up to 36 months, the material, whilst being impermeable to oxygen, will always end up allowing some oxygen through. According to experience, it is moreover difficult to ensure an oxygen replacement level that is close to 100%.

The present invention therefore addresses the problem of the stability of testosterone within SATDs, and more especially within SATDs of the matrix type. It has now been found in fact, and this being the case in surprising and completely unexpected manner, that the use of a desiccant agent makes it possible to limit the formation of oxidation impurities and hydroxylation impurities.

The present invention accordingly relates to the use of a desiccant agent in limiting the degradation of testosterone present within a self-adhesive transdermal device contained in a substantially sealing packaging.

A desiccant agent is understood to be any product having a strong affinity for water and capable of fixing the water contained in the internal atmosphere of the packaging or the water capable of penetrating into said packaging.

Degradation of testosterone is understood to be the formation of oxidation impurities and/or hydroxylation impurities of said testosterone.

It has accordingly been possible to note that employing a desiccant agent capable of dehydrating the adhesive matrix makes it possible, in surprising manner, to substantially limit the formation of oxidation derivatives and hydroxylation derivatives on prolonged storage of the SATD.

The use according to the present invention is understood to be the provision of a desiccant agent within a sachet-type packaging, preferably sealing against water-vapor and oxygen, which packaging contains the SATD comprising the testosterone.

The term “packaging” is understood to refer to an assemblage formed by the organization of materials of any kind intended to contain and protect the SATD during its handling, transport and storage.

“Substantially sealing” means that said packaging substantially counters the passage of oxygen and water vapor from the atmosphere into the interior of said packaging, this being the case throughout storage of the packaged SATD.

The desiccant agent preferably is physically dissociated from the SATD.

The desiccant agent may be any type of desiccant agent known and used in the pharmaceutical industry such as those used in tubes of tablets. For example, it may be silica gel or molecular sieve.

It preferably refers to molecular sieves which are most frequently composed of sodium or potassium salts of aluminum silicate, or oxides of silica, magnesium, sodium or of aluminum, for example. These substances are presented in the form of a very fine powder whose available size is between 1 and 10 Ä inclusive.

The desiccant agent is preferably not intended to be joined to the SATD but rather to be physically independent thereof. By the same token, this desiccant agent may be associated with the packaging and be joined thereto or physically independent thereof. For example, the desiccant agent may be provided, within the packaging containing the SATD, in the form of a free or adhesively fixed, porous sachet containing said desiccant agent. It could also refer, for example, to a free or adhesively fixable desiccant label comprising the desiccant agent sandwiched between an adhesive support and a porous covering sheet, for example of non-woven material. Such a label may accordingly be adhesively fixed to the inside of the sealing packaging containing the SATD. This label may be physically independent of the SATD as specified hereinbefore, although it is also feasible for it to be adhesively fixed to the outer surface of the support layer of the SATD, for example. Accordingly, as an especially suitable desiccant there may be mentioned the DESIMAX® product marketed by the company MULTISORB Technologies. This refers to a moisture-absorbent adhesive label which is very thin and which accordingly may be simply provided on the inside of a sealing packaging containing a testosterone SATD. Another type of especially suitable desiccant consists of a label made of polymers within which the desiccant agent proper is distributed as homogeneously as possible.

The testosterone-containing SATDs may also be packaged in a packaging of very low permeability such as a blister comprising a thermoformed tray of plastics material and a peelable lid in the form of a flexible, heat-sealed, aluminum/plastics, or preferably all-aluminum, sheet. The desiccant product may be provided within such packagings, in the form of a label adhesively fixed, preferably, to an inside surface of the blister, either on the tray or on the peelable lid. In the case of blister-type packaging, preference will be given to using aluminum both for the tray and for the lid, this being the case in order to obtain a degree of sealing against water-vapor that is as high as possible.

The SATDs in question may also be packaged in a sealing sachet formed by assembling multilayer films heat-sealed at their edges. Suitable multilayer films are those customarily used in the pharmaceutical industry; by way of example there may be mentioned paper/polyethylene/aluminum composites.

It is also possible to provide, within such packagings, either a porous sachet containing a desiccant agent or an optionally self-adhesive desiccant label. The presence of desiccant accordingly makes it possible to stabilize the testosterone contained in the SATD for storage periods of up to 12 months, even 24 months, or moreover even 36 months, with only very low formation of androstenedione type degradation products being found compared to a control without desiccant.

In a particular embodiment of the present invention, the desiccant agent may form an integral part of the packaging material. In this case it could be accordingly disposed in the heat-sealing zone between the multilayer films in the case of a sachet or between the peelable lid and the tray in the case of a blister.

The testosterone-containing transdermal systems to which the present invention relates are any type of SATD. However, the present invention relates more especially to devices that are referred to as matrix devices. As an example of a matrix SATD containing testosterone as active substance there may be mentioned the Patent Application FR 2793689 in the name of the Applicant. That application describes an SATD comprising, in the matrix layer, an acrylic-type polymer having acid functionality. The acid functionality results from the presence of acrylic acid among the base monomers; it is therefore to be understood that that self-adhesive polymer used in the matrix layer has free carboxylic acid side groups. In view of the acid character of that matrix material, which a priori is not favorable to maximum stability of the testosterone, the addition of polyvinylpyrrolidone made it possible to stabilize said testosterone physically. However, again due to the presence of those acid groups, it was found that the chemical stability of the testosterone in the long term was not optimal. The problem for the present invention is moreover to ensure the long-term chemical stability of the testosterone contained in a matrix SATD, especially in a matrix-type SATD in which the matrix layer comprises a self-adhesive polymer having acid functionality.

The present invention is accordingly aimed at stabilizing the testosterone contained in an SATD, especially a matrix-type SATD, preferably comprising at least one polymer having acid functionality.

In the context of the present invention, testosterone is understood to be testosterone as such or one of its derivatives. According to the present invention, derivatives of testosterone are understood to be not only its esters such as, for example, the acetate, enanthate, propionate, isobutyrate, undecanoate and cypionate forms but also derivatives such as those having a substituent at least in the 6-a or 7-a position. There may be especially mentioned, for example, 7-a-methyl-testosterone, 7-a-methyl-19-nortestosterone, 7-a-methyl-11b-hydroxy-testosterone, 7-a-17-dimethyltestosterone and 7-a,17-dimethyl-11b-hydroxytestosterone.

The use of a desiccant in association with a testosterone-containing SATD in accordance with the present invention has accordingly made it possible to considerably improve the chemical stability of the testosterone contained in such a device. Accordingly, for example when a desiccant of the DESIMAX® type is associated with a transdermal device such as that described in the French Patent Application FR2793689, it has been possible to observe an androstenedione content that is reduced by half and the other impurities being divided by a factor that is close to 4, this being the case relative to a control without desiccant, on storage for 30 months at 25° C./60% Relative Humidity (RH). Accordingly, as a corollary, the decrease in the testosterone content due to its degradation is, in the presence of a desiccant agent within the packaging during storage, reduced by a factor of 2, relative to the same control without desiccant.

The present invention is especially unexpected and surprising because it does indeed seem difficult to explain the limiting of oxidation and hydroxylation of the testosterone by virtue of the presence of a desiccant within the packaging containing the testosterone SATD. One hypothesis that has been advanced is that the complete dehydration of the adhesive matrix containing the testosterone makes it possible to significantly reduce the activity of the residual water within that matrix and, by the same token, reduces the kinetics of oxidation and hydroxylation product formation.

As stated hereinbefore, the present invention relates more especially to the chemical stabilization of the testosterone contained in a matrix-type self-adhesive transdermal device containing at least one polymer having acid functionality. The presence of desiccant accordingly seems to counter the deleterious effect especially of the acid functions in the polymer.

Finally, another problem for the present invention is to provide a pharmaceutical composition that is presented in the form of a self-adhesive transdermal device containing testosterone and provided in a sealing packaging containing, together therewith, a desiccant agent, said composition being characterized in that, after 12 months of storage at 25° C. and 60% Relative Humidity, preferably 24 months, even more preferably 36 months, the amount of androstenedione contained in the device is less than 3% by weight, or even less than 2% by weight, of the amount of testosterone, this also being the case for the other degradation impurities.

The transdermal device is, more preferably, a matrix-type device containing at least one self-adhesive acid-function polymer. Accordingly, said self-adhesive acid-function polymer is preferably a polymer of the monomer (meth)acrylic acid and at least one monomer selected from the group consisting of the monomers C1-C6 alkyl (meth)acrylate, vinyl acetate, glycidyl (meth)acrylate and 2-hydroxy(C1-C6 alkyl) (meth)acrylate. More preferably still, it can be a polymer of the monomer (meth)acrylic acid and at least one monomer selected from the group consisting of the monomers methyl (meth)acrylate, butyl (meth)acrylate, 2-ethylhexyl (meth)acrylate, vinyl acetate, glycidyl (meth)acrylate and 2-hydroxyethyl (meth)acrylate. Such an acrylic polymer is, for example, DUROTAK® 387-2052 or 87-2052 from the National Starch Company.

The present invention accordingly makes it possible to ensure the storage of a testosterone-containing SATD packaged in a sealing packaging, this being the case by virtue of a desiccant agent within that packaging. This makes it possible for testosterone-containing SATDs to be kept at a temperature of the order of 25° C. and at an RH of the order of 60%, for periods of up to 12 months, even 24 months, or even moreover 36 months, this being the case without finding that the androstenedione content threshold of about 3% by weight of the amount of testosterone is exceeded.

Such an invention is especially useful and simple to put into practice. In order to further illustrate the advantages of the present invention, exemplifying embodiments and stability tests are described hereinbelow.

### EXAMPLE 1

Results obtained with a 0.145 g self-adhesive desiccant label (Desimax®) on pilot batches of testosterone-based SATDs
A (% androstenedione, % by weight relative to the amount of testosterone)
O (% other impurities, % by weight relative to the amount of testosterone)

| | | months | | | | | |
|---|---|---|---|---|---|---|---|
| 25° C./60% RH | | 1 | 6 | 12 | 18 | 24 | 30 |
| CM851E01 60 cm² | A | 0.21 | 0.43 | 0.54 | 0.71 | 1.09 | 1.16 |
| (with desiccant) | O | 0.15 | 0.17 | 0.51 | 0.49 | 0.63 | 0.73 |
| CM852E02 60 cm² | A | 0.18 | 0.39 | 0.63 | 0.75 | 1.03 | 1.05 |
| (with desiccant) | O | 0.13 | 0.17 | 0.49 | 0.52 | 0.59 | 0.69 |

| | | months | | | | | |
|---|---|---|---|---|---|---|---|
| 30° C./70% RH | | 1 | 6 | 12 | 18 | 24 | 30 |
| CM851E01 60 cm² | A | 0.21 | 0.67 | 1.0 | 1.62 | 2.38 | 2.93 |
| (with desiccant) | O | 0.15 | 0.73 | 0.70 | 0.73 | 0.99 | 1.19 |
| CM852E02 60 cm² | A | 0.18 | 0.65 | 1.13 | 1.58 | 2.16 | 2.85 |
| (with desiccant) | O | 0.13 | 0.43 | 0.73 | 0.77 | 0.99 | 1.15 |

the compositions CM851E01 and CM852E02 are 2 batches of testosterone-based SATDs having the following composition:
   o 69%, by weight, of a self-adhesive polymer of the monomers acrylic acid, 2-ethylhexyl acrylate, vinyl acetate and butyl acrylate, this polymer having been cross-linked and having an acid index of between 10 and 70 inclusive and a glass transition temperature of between −100° C. and −10° C. inclusive (DUROTAK 387-2052)
   15%, by weight of the SATD, of polyvinylpyrrolidone having a molecular weight of between 44000 and 54000 inclusive,
   11%, by weight of the SATD, of N,N-diethyl-m-toluamide,
   5%, by weight of the SATD, of testosterone

The SATDs are packaged in a sealing sachet composed of two heat-sealed triple-layer Paper/Aluminum/PolyEthylene sheets. The desiccant label is provided on an inner surface of the sachet.

According to Example 1, the use of the DesiMax® desiccant label in accordance with the present invention makes it possible to significantly reduce the formation of impurities; for example, at 12 months 30° C./65% RH, and in the absence of Desimax, the mean values of androstenedione and other impurities are, respectively, 1.9% and 2.17% of the amount of testosterone, by weight. Under the same storage conditions and after the same time period, in the presence of the DesiMax® desiccant label, the amounts of androstenedione are reduced by half and the other impurities by about 70%.

### EXAMPLE 2

Results obtained with a 0.145 g Desimax® self-adhesive desiccant label on industrial batches of testosterone-based SATDs
A (% androstenedione, in terms of % of the amount of testosterone by weight)
O (% other impurities, in terms of % of the amount of testosterone by weight)

months

| 25° C./60% RH | | 0 | 3 | 6 | 9 |
|---|---|---|---|---|---|
| 7043524 45 cm² | A | 0.36 | 0.73 | 0.97 | 1.15 |
| (without desiccant) | O | 0.56 | 1.21 | 1.09 | 1.84 |
| 7043534 60 cm² | A | 0.38 | 0.58 | 0.74 | 1.35 |
| (without desiccant) | O | 0.58 | 1.04 | 1.12 | 2.05 |
| 7043554 45 cm² | A | 0.37 | 0.53 | 0.65 | 0.68 |
| (with desiccant) | O | 0.52 | 0.7 | 0.75 | 0.86 |
| 7043564 60 cm² | A | 0.4 | 0.49 | 0.56 | 0.62 |
| (with desiccant) | O | 0.67 | 0.72 | 0.72 | 0.85 |

months

| 30° C./65% RH | | 0 | 3 | 6 | 9 |
|---|---|---|---|---|---|
| 7043524 45 cm² | A | 0.36 | 0.79 | 0.97 | 1.15 |
| (without desiccant) | O | 0.56 | 1.28 | 1.15 | 1.89 |
| 7043534 60 cm² | A | 0.38 | 0.49 | 1.62 | 0.62 |
| (without desiccant) | O | 0.58 | 0.99 | 1.57 | 1.37 |
| 7043554 45 cm² | A | 0.37 | 0.60 | 0.76 | 0.85 |
| (with desiccant) | O | 0.52 | 0.77 | 0.80 | 0.99 |
| 7043564 60 cm² | A | 0.4 | 0.49 | 0.63 | 0.79 |
| (with desiccant) | O | 0.67 | 0.78 | 0.84 | 0.98 |

the compositions 7043524, 7043534, 7043554 and 7043564 are testosterone-based SATDs corresponding to the compositions described according to Example 1 and packaged in a manner identical to that described in Example 1.

According to Example 2, the use of the DesiMax® desiccant label in accordance with the invention also makes it possible to improve the stability of SATDs produced on an industrial scale. For example, in the course of the first 9 months of stability testing at 25° C./60% RH, the presence of this label makes it possible to slow down the formation of the degradation products; the amounts of androstenedione and other impurities are reduced by half; the physical stability of the SATDs is excellent.

### EXAMPLE 3

Results obtained with a 0.145 g Desimax® desiccant label on pilot batches of testosterone-based SATDs; comparison of water contents, in terms of % of the SATD by weight.

| 25° C./60% RH | Water content | 1 | 12 | 18 | 24 | 30 |
|---|---|---|---|---|---|---|
| CM851E01 60 cm² | in the SATD | 0.16 | 0.12 | 0.09 | 0.05 | 0.12 |
| (with desiccant) | in Desimax | 7.50 | 8.85 | 8.15 | 8.86 | 9.14 |
| CM852E02 60 cm² | in the SATD | 0.19 | 0.07 | 0.06 | 0.10 | 0.09 |
| (with desiccant) | in Desimax | 7.30 | 8.76 | 6.44 | 7.42 | 5.85 |

| 30° C./70% RH | Water content | 1 | 12 | 18 | 24 | 30 |
|---|---|---|---|---|---|---|
| CM851E01 60 cm² | in the SATD | 0.16 | 0.12 | 0.12 | 0.09 | 0.07 |
| (with desiccant) | in Desimax | 7.50 | 6.79 | 6.25 | 6.84 | 8.32 |
| CM852E02 60 cm² | in the SATD | 0.19 | 0.07 | 0.07 | 0.09 | 0.13 |
| (with desiccant) | in Desimax | 7.30 | 8.36 | 7.83 | 7.80 | 8.82 |

According to Example 3, the use of the DesiMax® label in accordance with the invention makes it possible to almost completely dehydrate the SATD; the initial mean water content before packaging (0.8%) drops rapidly and reaches a level lower than 0.1%.

1-11. (canceled)

12. A method of limiting degradation of testosterone present within a self-adhesive transdermal device comprising, combining a desiccant agent and the self-adhesive transdermal device in a substantially sealing packaging.

13. The method of claim 12, wherein the desiccant agent is physically independent of the self-adhesive transdermal device.

14. The method of claim 12, wherein the desiccant agent is physically dissociated from the packaging.

15. The method of claim 12, wherein the desiccant agent is associated with the packaging.

16. The method of claim 12, wherein the desiccant agent is adhesively fixed to an inner surface of the packaging.

17. The method of claim 12, wherein the desiccant agent is selected from molecular sieves and silica gels.

18. The method of claim 17, wherein the molecular sieves are selected from sodium or potassium salts of aluminum silicate, and oxides of silica, of magnesium, of sodium, and of aluminum.

19. The method of claim 12, wherein the desiccant agent is included in a porous sachet.

20. The method of claim 12, wherein the desiccant agent is included in an optionally self-adhesive label.

21. The method of claim 20, wherein the self-adhesive label comprises a desiccant agent sandwiched between an adhesive support and a porous covering sheet.

22. The method of claim 20, wherein the self-adhesive label comprises at least one polymer within which a desiccant agent is distributed.

23. A pharmaceutical product comprising, a substantially sealing packaging containing, a self-adhesive transdermal device which includes a pharmaceutically effective amount of testosterone, and a desiccant agent which limits the degradation of testosterone present within the self-adhesive transdermal device.

24. The pharmaceutical product of claim 23, wherein the desiccant agent is included in the form of a free or adhesively fixed, porous sachet containing the desiccant agent.

25. The pharmaceutical product of claim 24, wherein the desiccant agent is adhesively fixed to an inner surface of the packaging.

26. The pharmaceutical product of claim 24, wherein the desiccant agent is provided in an optionally self-adhesive label.

27. The pharmaceutical product of claim 26, wherein the self-adhesive label comprises a desiccant agent sandwiched between an adhesive support and a porous covering sheet.

28. The pharmaceutical product of claim 26, wherein the self-adhesive label comprises at least one polymer within which a desiccant agent is distributed.

29. The pharmaceutical product of claim 23, wherein the desiccant agent is selected from molecular sieves and silica gels.

30. The pharmaceutical product of claim 29, wherein the molecular sieves are selected from sodium or potassium salts of aluminum silicate, and oxides of silica, of magnesium, of sodium, and of aluminum.

31. The pharmaceutical product of claim 23, wherein the self-adhesive transdermal device is a matrix-type device.

32. The pharmaceutical product of claim 31, wherein a matrix layer of the matrix-type device comprises a self-adhesive polymer with acid functionality.
